(19) United States
(12) Patent Application Publication
(10) Pub. No.: US 2020/0060514 A1
(43) Pub. Date: Feb. 27, 2020

(54) ENDOSCOPE AND METHOD OF MANUFACTURING ENDOSCOPE

(71) Applicant: FUJIFILM Corporation, Tokyo (JP)

(72) Inventor: Toshio SAKAMOTO, Kanagawa (JP)

(73) Assignee: FUJIFILM Corporation, Tokyo (JP)

(21) Appl. No.: 16/546,320

(22) Filed: Aug. 21, 2019

(30) Foreign Application Priority Data
Aug. 24, 2018 (JP) 2018-157335

(51) Int. Cl. A61B 1/00; G02B 23/24; H04N 7/18

(52) U.S. Cl. CPC A61B 1/0011; A61B 1/00096; G02B 23/2484; H04N 7/18; G02B 23/2423

(57) ABSTRACT

Provided are an endoscope and a method of manufacturing an endoscope capable of positioning an imaging element and a lens assembly with high accuracy, forming an optical image with high accuracy on an image reception surface of the imaging element to obtain a high-quality image with little loss of to image, defocusing, and the like, performing easy assembling, and firmly joining members.

The endoscope has an imaging element; a lens assembly that forms an optical image on an image reception surface of an imaging element; a cable that is connected to a connecting terminal provided on a surface opposite to the image reception surface of the imaging element; a case that protects a joined part between the connecting terminal of the imaging element and the cable; and a holder that holds the lens assembly and is joined to the case. The case has an opening part penetrating in an arrangement direction of the imaging element and the cable, and is disposed such that an opening surface on the imaging element side is in contact with an end surface of the holder. The imaging element is disposed such that the imaging element abuts against at least one of inner wall surfaces of the opening part of the case in a lateral surface and the image reception surface abuts against the end surface of the holder.

### CROSS-REFERENCE TO RELATED APPLICATIONS

This application claims priority under 35 U.S.C. § 119(a) to Japanese Patent Application No. 2018-157335 filed on Aug. 24, 2018. The above application is hereby expressly incorporated by reference, in its entirety, into the present application.

### BACKGROUND OF THE INVENTION

### 1. Field of the Invention

The invention relates to an endoscope and a method of manufacturing an endoscope.

### 2. Description of the Related Art

Generally, imaging devices to be mounted on distal end parts of insertion parts of endoscopes comprise a solid-state imaging element (image sensor), a lens assembly that forms an optical image on an image reception surface of an individual imaging element, and a cable connected to the individual imaging element.

In the individual imaging element and the lens assembly, it is necessary to accurately position optical axes of lenses at the center of the image reception surface of the individual imaging element in order for the lens assembly to form the optical image on the image reception surface of the individual imaging element. For that reason, the individual imaging element and the lens assembly are fixed to one frame member and the relative positions of the individual imaging element and the lens assembly are fixed.

For example, JP2014-087705A discloses an imaging device to be mounted on a distal end part of an electronic endoscope. The imaging device disclosed in JP2014-087705A has an objective optical unit having an objective lens group that constitutes an objective optical system, and an imaging unit having an imaging element and the like. JP2014-087705A discloses that the objective optical unit has a rear part of an objective lens frame fitted and fixed to a distal end side of the imaging element frame and has the imaging element disposed on a rear end side of the imaging element frame such that light enters the center of a light-receiving part of the imaging element to form a subject image (refer to FIG. 3 and paragraphs [0019] and [0020]).

Additionally, JP2012-183330A discloses an imaging device comprising an imaging element, a circuit board that is connected to the imaging element and has electronic components mounted on thereon, a plurality of signal line connecting part that are formed on the circuit board and have a plurality of signal lines of a composite signal cable connected thereto, and a plurality of ground signal line binding parts formed by the plurality of signal lines. The imaging device disclosed in JP2012-183330A has an objective optical unit having an objective lens group as an objective optical system, and an imaging element package. JP2012-183330A discloses that a rear end side of an objective lens frame of the objective optical unit is internally fitted and fixed to a distal end side of an imaging element frame, and an imaging element package is provided on a rear end side of the imaging element frame.

Additionally, JP2012-064883A discloses an imaging device comprising an imaging element chip having an imaging element on a first principal surface and having an external connection terminal connected to the imaging element on a second principal surface, a wiring board that has a wiring layer connected to the external connection terminal and is disposed within a projection plane of the imaging element chip by being bent at a bent part, and a joining layer that is disposed within the projection plane of the imaging element chip and joins the imaging element chip and the wiring board to each other. JP2012-064883A discloses that a frame part for housing an optical system, and the imaging element are disposed inside a tubular shield frame (refer to FIG. 19).

### SUMMARY OF THE INVENTION

The imaging devices disclosed in JP2014-087705A and JP2012-183330A has a configuration in which the imaging element frame is cylindrical and has a shape having an inner flange part on an inner peripheral surface of a tube, and the imaging unit (imaging element) is positioned by making the image reception surface side of the imaging unit abut against this inner flange part and making a lateral surface of the imaging unit abut against the inner peripheral surface of the imaging element frame.

Since the imaging device of the endoscope is very small, respective parts that constitute the imaging device are also small. For that reason, a rounded part of unignorable size may be formed at a joint between the inner flange part of the imaging element frame and the inner peripheral surface of the tube. Meanwhile, the image reception surface side of the imaging element is covered with a cover glass, and an end edge of the cover glass is cut out at an acute angle. For that reason, in a configuration in which the imaging element is positioned using the tubular imaging element frame having the inner flange part, the end edge of the imaging element (cover glass) may come into contact with the rounded part between the inner flange part of the imaging element frame and the inner peripheral surface of the tube, the image reception surface of the imaging element may be inclined with respect to the optical axis of the lens assembly, and a center position of the image reception surface of the imaging element may shift from the optical axis of the lens assembly, or the distance between the imaging element and the lens assembly may shift. Therefore, it is impossible to position the imaging element and the lens assembly with high accuracy. Accordingly, there are problems that an optical image is not formed with high accuracy on the image reception surface of the imaging element, and image quality degrades due to loss of an image, defocusing, and the like.

Additionally, in the imaging device disclosed in JP2012-064883A, the frame part (lens assembly) that houses the optical system is disposed inside the tubular shield frame (case). Therefore, it is difficult to accurately and sufficiently fix the lens assembly and the case. In a case where the joining between the lens assembly and the case is weak, there is a concern that a joined portion between the lens assembly and the case may be damaged due to external force, humidity, disinfectant, or the like.

For example, although it is considered that the lens assembly and the case are fixed to each other with an adhesive, recent disinfectant is powerful for infection prevention, and the adhesive (resin) may be deteriorated.

Additionally, although it is also considered that the lens assembly and the case are joined to each other by laser welding or the like, there is a concern that the lens assembly and the case may be deformed. Additionally, although thickening is considered for deformation avoidance, there is a problem that dimensions may become large and the insertion part cannot be made thin.

An object of the invention is to solve such a problem and to provide an endoscope and a method of manufacturing an endoscope capable of positioning an imaging element and a lens assembly with high accuracy, forming an optical image with high accuracy on an image reception surface of the imaging element to obtain a high-quality image with little loss of to image, defocusing, and the like, performing easy assembling, and firmly joining members.

The invention solves the object by the following configurations.

[1] An endoscope comprising an imaging element; a lens assembly that forms an optical image on an image reception surface of an imaging element; a cable that is connected to a connecting terminal provided on a surface opposite to the image reception surface of the imaging element; a case that protects a joined part between the connecting terminal of the imaging element and the cable; and a holder that holds the lens assembly and is joined to the case, the case having an opening part penetrating in an arrangement direction of the imaging element and the cable, and being disposed such that an opening surface on the imaging element side is in contact with an end surface of the holder, and the imaging element being disposed such that the imaging element abuts against at least one of inner wall surfaces of the opening part of the case in a lateral surface and the image reception surface abuts against the end surface of the holder.

[2] The endoscope according to [1] in which the lateral surface of the imaging element abuts against two adjacent inner wall surfaces of the opening part of the case.

[3] The endoscope according to [1] or [2] in which the lateral surface of the imaging element abuts against two facing inner wall surfaces, and one inner wall surface adjacent to the two facing inner wall surfaces of the opening part of the case.

[4] The endoscope according to any one of [1] to [3] in which the holder has at least one claw part to be locked to an outer wall surface of the case.

[5] The endoscope according to any one of [1] to [4] in which the holder has a hole part that penetrates in the arrangement direction of the imaging element and the cable and holds the lens assembly.

[6] The endoscope according to [5], further comprising a cylindrical pipe-like member into which the cable is to be inserted, the pipe-like member being disposed within the opening part of the case, and a central axis of a circle inscribed on a cross-sectional shape of the opening part of the case at a disposition position of the pipe-like member, a central axis of an external diameter of the pipe-like member, and a central axis of the hole part of the holder coinciding with each other.

[7] The endoscope according to claim [6] in which a diameter of the circle inscribed on the cross-sectional shape of the opening part of the case at the disposition position of the pipe-like member and the external diameter of the pipe-like member coincide with each other.

[8] The endoscope according to any one of [1] to [7] in which the cross-sectional shape of the opening part of the case at the disposition position of the imaging element is a rectangular shape.

[9] A method of manufacturing the endoscope according to any one of [1] to [8], comprising a joining step of joining a case and a holder to each other; an imaging element disposing step of disposing an imaging element such that the imaging element abuts against at least one of inner wall surfaces of an opening part of the case and such that an image reception surface abuts against an end surface of a holder, after the joining step; and a lens assembly disposing step of disposing a lens assembly on the holder, in the joining step, the case and the holder are joined to each other in a state where a pin is inserted through the opening part of the case and a hole part of the holder and one opening surface of the opening part of the case abuts against an end surface of the holder, and a diameter of the pin, a diameter of the hole part of the holder, and a diameter of a circle inscribed on a cross-sectional shape in at least a portion of the opening part of the case coincide with each other.

[10] The method of manufacturing the endoscope according to [9], further comprising a cable connecting step of connecting the cable to the connecting terminal of the imaging element before the imaging element disposing step.

According to the invention, it is possible to provided the endoscope and the method of manufacturing an endoscope capable of positioning the imaging element and the lens assembly with high accuracy, forming the optical image with high accuracy on the image reception surface of the imaging element to obtain a high-quality image with little loss of to image, defocusing, and the like, performing easy assembling, and firmly joining members.

### BRIEF DESCRIPTION OF THE DRAWINGS

FIG. 1 is a view conceptually illustrating an example of an endoscope system using the endoscope of the invention.

FIG. 2 is a perspective view conceptually illustrating an example of the endoscope of the invention.

FIG. 3 is a front view of the endoscope illustrated in FIG. 2.

FIG. 4 is a side view of the endoscope illustrated in FIG. 2.

FIG. 5 is a rear view of the endoscope illustrated in FIG. 2.

FIG. 6 is a perspective view of a holder.

FIG. 7 is a perspective view of a case.

FIG. 8 is a cross-sectional view of a curved surface of a case.

FIG. 9 is a perspective view illustrating a junction state between the case and a holder.

FIG. 10 is a cross-sectional view illustrating another example of the curved surface of the case.

FIG. 11 is a view for illustrating an example of a method of manufacturing the endoscope of the invention.

### DESCRIPTION OF THE PREFERRED EMBODIMENTS

Hereinafter, embodiments of an endoscope the invention will be described with reference to the drawings.

Although the description of constituent elements to be described below is made on the basis of the representative embodiments of the invention, the invention is not limited to such embodiments. In the drawings of the present specification, scales of respective parts are appropriately changed and illustrated in order to facilitate visual recognition.

In addition, numerical ranges expressed using “to” in the present specification mean ranges including numerical values described before and after “to” as lower limits and upper limits.

Endoscope

An endoscope of the invention has, an imaging element, a lens assembly that forms an optical image on an image reception surface of the imaging element; a cable that is connected to a connecting terminal provided on a surface opposite to the image reception surface of the imaging element; a case that protects a joined part between the connecting terminal of the imaging element and the cable; and a holder that holds the lens assembly and is joined to the case, the case has an opening part that penetrates in an arrangement direction of the imaging element and the cable, and is disposed such that an opening surface on the imaging element side is in contact with an end surface of the holder, and the imaging element is disposed such that a lateral surface abuts against at least one of inner wall surfaces of the opening part of the case and the image reception surface abuts against the end surface of the holder.

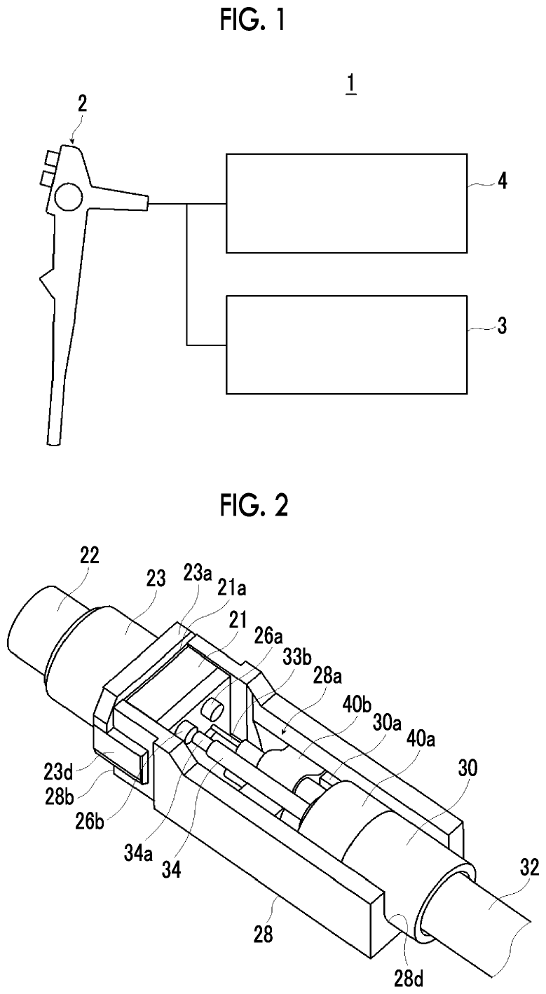

An example of an endoscope system having the endoscope of the invention is conceptually illustrated in FIG. 1.

An endoscope system 1 comprises an endoscope 2, a light source unit 3, and a processor unit 4. The endoscope 2 has the same configuration as general endoscopes except for the portion of an imaging device 20 to be described below. The endoscope 2 comprises an insertion part to be inserted into a subject, an operating part connected to the insertion part, and a universal cord extending from the operating part, and the insertion part is constituted of a distal end part, a bending part connected to the distal end part, and a flexible part that connects the bending part and the operating part to each other.

The distal end part is provided with an illumination optical system that emits illumination light for illuminating an observation region, an imaging device and an imaging optical system that image the observation region, and the like. The bending part is configured to be bendable in a direction orthogonal to the longitudinal axis of the insertion part, and a bending operation of the bending part is performed in the operating part. Additionally, the flexible part is configured to be relatively flexible so as to be deformable along the shape of an insertion path of the insertion part.

The operating part is provided with a button that operates an imaging operation of the imaging device of the distal end part, a knob that operates the bending operation of the bending part, and the like. Additionally, the operating part is provided with an introduction port into which treatment tools, such as an electric scalpel, are introduced, and a treatment tool channel which reaches the distal end part from the introduction port and through which treatment tools, such as forceps, are inserted is provided inside the insertion part.

A terminal of the universal cord is provided with a connector, and the endoscope 2 is connected via the connector to the light source unit 3 that creates the illumination light emitted from the illumination optical system of the distal end part, and the processor unit 4 that processes video signals acquired by the imaging device of the distal end part. The processor unit 4 processes the input video signals to create image data of the observation region, and displays and records the created image data on a monitor.

A light guide and an electric wire group (cable bundle) are housed inside the insertion part, the operating part, and the universal cord. The illumination light created in the light source unit 3 is guided to the illumination optical system of the distal end part via the light guide, and signals and electrical power are transmitted via the electric wire group between the imaging device of the distal end part and the processor unit 4.

A distal end hard part of the endoscope 2 is provided with the imaging device 20, and a distal end part of the treatment tool channel, and is also provided with the illumination optical system or the like that emits the illumination light guided from the light source unit 3 via the light guide.

A case 28, which holds an image sensor (imaging element) 21 of the imaging device 20, and a holder 23, which holds a lens assembly 22, are housed in a housing hole formed in the distal end hard part made of, for example, metallic materials, such as a stainless steel material, and are fixed to the distal end hard part. The distal end part of the treatment tool channel, and the illumination optical system are also respectively housed in the housing hole formed in the distal end hard part, and are fixed to the distal end hard part.

An image reception surface 21a of the image sensor 21 held by the case 28 fixed to the distal end hard part is disposed substantially perpendicularly to a longitudinal axis of the insertion part.

FIGS. 2 to 5 illustrate the configuration of the imaging device mounted on the distal end hard part of the insertion part of the endoscope 2 of the invention.

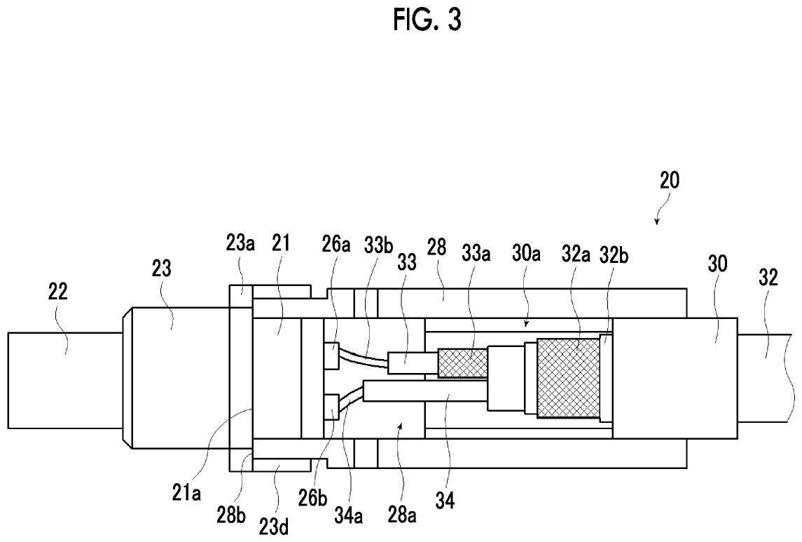

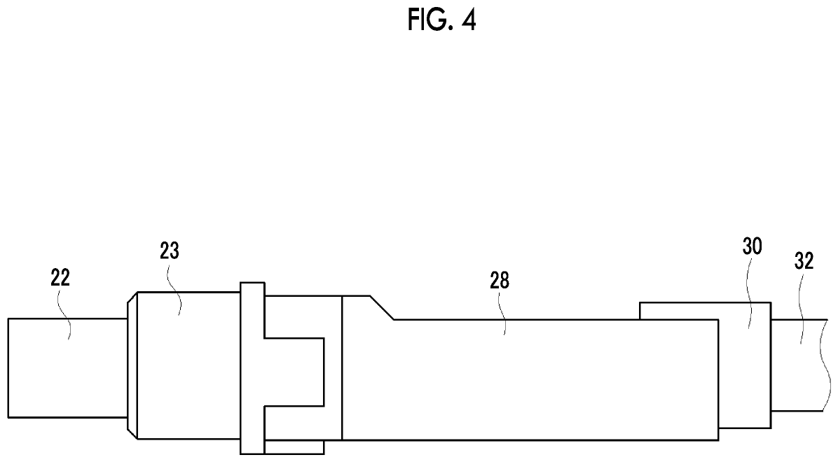

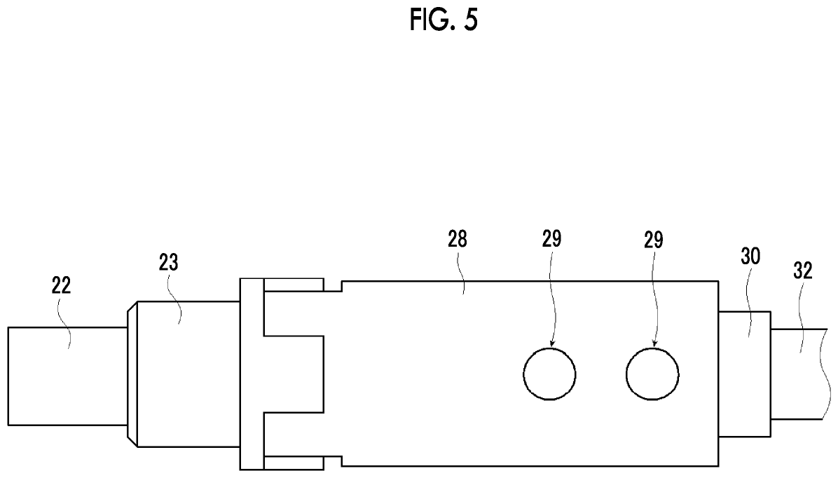

FIG. 2 is a perspective view conceptually illustrating an example of the imaging device 20 of the endoscope 2. FIG. 3 is a front view of the imaging device 20 illustrated in FIG. 2. FIG. 4 is a side view of the imaging device 20 illustrated in FIG. 2. FIG. 5 is a rear view of the imaging device 20 illustrated in FIG. 2. In addition, illustration of the conductive joining members 40a and 40b is omitted in FIG. 3.

The imaging device 20 illustrated in FIGS. 2 to 5 comprises the image sensor (imaging element) 21, the lens assembly 22, the holder 23, a cable bundle 32, a pipe-like member 30, the case 28, and the conductive joining members 40. The conductive joining members 40 are at least one of solder, metal braze, or metal paste.

The mage sensor (imaging element) 21 is a well-known image sensor that of which the image reception surface 21a is disposed to intersect a longitudinal direction of the insertion part, and photoelectrically converts an optical image formed on the image reception surface 21a. In addition, as the image sensor 21, well-known image sensors, such as a charge coupled device (CCD) image sensor and a complementary metal-oxide semiconductor (CMOS) image sensor are available.

In the image sensor 21 to be used for endoscopes, the external diameter of the image sensor 21 as seen in a normal direction of the image reception surface 21a is equal to less than 1 mm.

A plurality of connecting terminals which signals and electrical power are input to and output from are provided on a back surface opposite to the image reception surface 21a of the image sensor 21. In addition, in FIG. 2 and the like, some (two) of the plurality of connecting terminals are designated by reference signs 26a and 26b, and four connecting terminals are provided in examples illustrated in FIG. 2 and the like.

Cables to be described below are connected to the plurality of connecting terminals, respectively.

Additionally, the image sensor 21 may have a glass cover or the like that covers the image reception surface 21a side.

The lens assembly 22 forms the optical image (subject image) on the image reception surface 21a of the image sensor 21. The lens assembly 22 has, for example, the imaging optical system in which optical axes of one or more lenses are arranged to coincide with each other, and a lens barrel that houses the imaging optical system. As the lens assembly 22, well-known configurations are appropriately available as long as the optical image can be formed on the image reception surface 21a of the image sensor 21.

The lens assembly 22 is disposed such that an optical axis of the imaging optical system perpendicularly intersects the center of the image reception surface 21a of the image sensor 21.

The holder 23 is a member that holds the lens assembly 22 and is joined to the case 28.

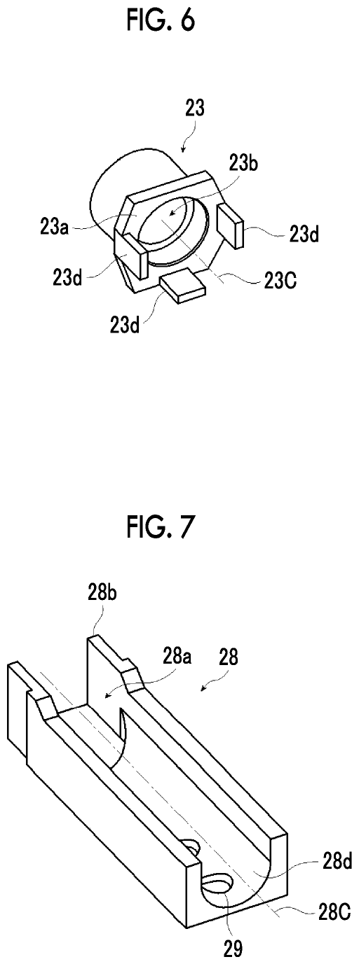

A perspective view of the holder 23 is illustrated in FIG. 6. In an example illustrated in FIG. 6, the holder 23 has a cylindrical shape having a hole part 23b of which both ends are open, and has a flange part 23a on one opening surface side. Additionally, claw parts 23d, which are erected in a direction perpendicular from the surface of the flange part 23a, are provided on three end edges of the flange part 23a. The claw parts 23d are locked to an outer wall surface of the case 28.

The hole part 23b of the holder 23 has substantially the same diameter as the external diameter of at least a portion of the lens assembly 22 (lens barrel), and has a size such that at least a portion of the lens assembly 22 is insertable therethrough. Accordingly, the hole part 23b holds the lens assembly 22 so as to be movable along the optical axis of the imaging optical system. The lens assembly 22 is moved within the hole part 23b such that the position of the image sensor 21 with respect to the imaging optical system is adjustable. The lens assembly 22 is fixed to the holder 23, for example, by an adhesive and the like after the image sensor 21 is positioned.

In addition, the cross-sectional shape of the hole part 23b of the holder 23 is not limited to the circular shape, and can be various shapes, such as a square shape, an oblong shape, and an elliptical shape.

The case 28 is a member that holds the image sensor 21 and protects joined parts between the connecting terminals of the image sensor 21 and the cables. Additionally, the case 28 is joined to the holder 23.

A perspective view of the case 28 is illustrated in FIG. 7. In an example illustrated in FIG. 7, the case 28 is a member of a hollow rectangular parallelepiped shape in which three faces are open. That is, the case 28 illustrated in FIG. 7 has a shape having one oblong plate-shaped member serving as a bottom surface, and two oblong plate-shaped members serving as two lateral surfaces erected from both end edges on a longitudinal side of the bottom surface. A space surrounded by the two lateral surfaces and the bottom surface is an opening part 28a of the case 28.

As illustrated in FIG. 2 and the like, the opening part 28a is open on both end sides in an arrangement direction of the image sensor 21 and the cables, and can also be said that the opening part 28a penetrates in the arrangement direction of the image sensor 21 and the cables.

Additionally, as illustrated in FIG. 5, a through-hole 29 is formed in the bottom surface of the case 28.

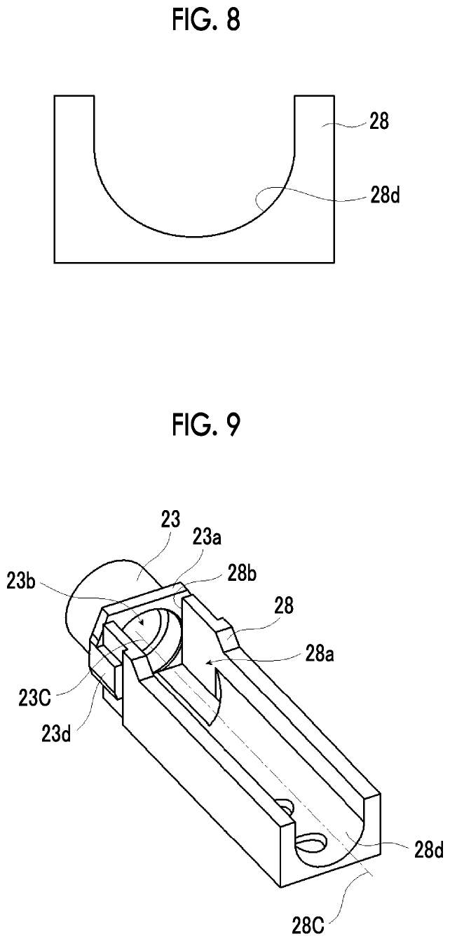

The opening part 28a of the case 28 has a substantially rectangular cross-sectional shape perpendicular to the longitudinal direction on one end surface 28b side in the longitudinal direction. On the other hand, the opening part 28a has a substantially semi-circular cross-sectional shape perpendicular to the longitudinal direction on the other end surface side in the longitudinal direction (refer to FIG. 8).

As illustrated in FIG. 2, the image sensor 21 is disposed on the end surface 28b side of a portion of the case 28 of which the cross-sectional shape of the opening part 28a is a substantially rectangular shape. The case 28 and the image sensor 21 are preferably joined to each other with an adhesive.

Additionally, the pipe-like member 30 through which the cable bundle 32 is inserted through is disposed in a portion (hereinafter, referred to as a curved surface 28d) of the case 28 of which the cross-sectional shape is a substantially semicircular shape. This point will be described below in detail.

Additionally, the holder 23 is joined to an end part of the case 28 on the image sensor 21 side.

Here, in the endoscope of the invention, as illustrated in FIG. 9, the end surface 28b of the case 28 on the side (the side where the cross-sectional shape is a substantially rectangular shape) where the image sensor 21 is disposed is disposed to abut against the flange part 23a of the holder 23. Additionally, as a preferred aspect, the claw parts 23d of the holder 23 are locked to an outer peripheral surface of the case 28.

Additionally, as illustrated in FIG. 2 and the like, the image sensor 21 is disposed within the opening part 28a of the case 28 such that the image reception surface 21a abuts against the flange part 23a of the holder 23. Additionally, in the illustrated example, the image sensor 21 is disposed such that three lateral surfaces thereof abut against three inner wall surfaces (the bottom surface and the two lateral surfaces) of the opening part 28a of the case 28, respectively.

As mentioned above, in the case of the configuration in which the image sensor is disposed and positioned so as to abut against the inner flange part of the imaging element frame, the end edge of the image sensor (the cover glass that covers the image reception surface of the image sensor) may come into contact with the rounded part between the inner flange part of the imaging element frame and the inner peripheral surface of the tube, the image reception surface of the image sensor may be inclined with respect to the optical axis of the lens assembly, and the center position of the image reception surface of the image sensor may shift from the optical axis of the lens assembly, or the distance between the image sensor and the lens assembly may shift. Therefore, there are problems that the image sensor and the lens assembly cannot be positioned with high accuracy, an optical image is not formed with high accuracy on the image reception surface of the imaging element, and image quality degrades due to loss of an image, defocusing, and the like.

Additionally, in the case of the configuration in which the holder (lens assembly) that houses the optical system inside the tubular case, it is necessary to join the holder and the case to each other inside the case. Therefore, there is a problem that it is difficult to accurately and sufficiently fix the holder and the case. In a case where the joining between the holder and the case is weak, there is a concern that a joined portion between the holder and the case may be damaged due to external force, humidity, disinfectant, or the like.

In a case where the joined portion is weak, for example, in a case where the holder and the case are fixed to each other with an adhesive, there is a concern that adhesive resin may deteriorate by the disinfectant. Additionally, although it is also considered that the holder and the case are joined to each other by laser welding or the like, there is a concern that the holder and the case may be deformed.

In contrast, the endoscope of the invention has a configuration in which the end surface 28b of the case 28 where the image sensor 21 is disposed abuts against the holder 23 (the flange part 23a of the holder 23), and the image reception surface 21a of the image sensor 21 abuts against the flange part 23a (end surface) of the holder 23, and at least one of the lateral surfaces of the image sensor 21 abuts against the inner wall surfaces of the opening part 28a of the case 28.

In this way, by forming the surface for positioning the image sensor 21 such that the end surface 28b of the case 28 and the flange part 23a of the holder 23 are made to abut against each other, the rounded part is not formed between the end surface 28b of the case 28 and the flange part 23a of the holder 23. Therefore, the image reception surface 21a of the image sensor 21 can be thoroughly brought into contact with the flange part 23a of the holder 23, and the lateral surfaces of the image sensor 21 can be reliably brought into contact with the inner wall surfaces of the case 28. Therefore, the image reception surface of the image sensor can be positioned perpendicularly to the optical axis of the lens assembly, the center position of the image reception surface of the image sensor in a surface direction of the image reception surface can be positioned with high accuracy at the position of the optical axis of the lens assembly, and the distance between the image sensor and the lens assembly can be positioned with high accuracy. Therefore, the optical image to be formed by the lens assembly can be formed with high accuracy on the image reception surface of the imaging element, and a high-quality image with no loss of the image, defocusing, and the like can be obtained.

Additionally, since the endoscope of the invention has a configuration in which the end surface 28b of the case 28 abuts against the holder 23, and the case 28 and the holder 23 are joined to each other, the case 28 and the holder 23 can be joined to each other from the outside of the case 28. For that reason, the holder and the case can be accurately and firmly fixed to each other. Therefore, it is possible to suppress that the joined portion between the holder and the case is damaged due to the disinfectant or the like, and the holder and/or the case can be prevented from being deformed.

Here, in the example illustrated in FIG. 2, the image sensor 21 has the configuration in which the three lateral surfaces abuts against the three inner wall surfaces of the opening part 28a of the case 28. That is, the configuration in which the three lateral surfaces of the image sensor 21 abut against two facing inner wall surfaces of the opening part 28a of the case 28, and one inner wall surface adjacent to the two inner wall surfaces is adopted. However, the invention is not limited to this, and a configuration in which at least one lateral surface of the image sensor 21 abuts against the inner wall surfaces of the opening part 28a of the case 28 may be adopted. For example, a configuration in which two lateral surfaces of the image sensor 21 abuts against two adjacent inner wall surfaces of the opening part 28a of the case 28 may be adopted.

As the number of lateral surfaces made to abut against the inner wall surfaces of the opening part 28a of the case 28 increases, the image sensor 21 can be positioned with high accuracy.

In addition, in a case where the image sensor 21 has the configuration in which the three lateral surfaces abut against the three inner wall surfaces of the opening part 28a of the case 28, the width between the two facing lateral surfaces of the opening part 28a of the case 28 is substantially the same as that of the width of the image sensor 21.

Additionally, as illustrated in FIG. 9, it is preferable that a central axis 23C of the hole part 23b of the holder 23 and a central axis 28C of a circle inscribed on the curved surface 28d of the case 28 coincide with each other.

Methods of joining the case 28 and the holder 23 to each other can be performed by a method using the above-described conductive joining member, and methods, such as welding and pressure welding.

As mentioned above, a cable included in the cable bundle 32 is electrically connected to each connecting terminal of the image sensor 21. The cable bundle 32 comprises one or more cables that are respectively connected to one or more connecting terminals provided on a surface opposite to the image reception surface 21a of the image sensor 21, and a collective shield wire that covers an outer periphery of the one or more cables.

In the example illustrated in FIGS. 2 to 5, the cable bundle 32 has four cables including a coaxial cable 33 and a single-axis cable 34, a collective shield wire 32a that covers an outer periphery of the four cables via an insulator with that encapsulates these four cables, and a protective covering (sheath) 32b that covers an outer periphery of the collective shield wire 32a.

As illustrated in FIG. 2, FIG. 3, and the like, the cable bundle 32 has the sheath 32b peeled at an end part thereof on the image sensor 21 side to expose the collective shield wire 32a and the four cables.

In the invention, the end part of the cable bundle on the image sensor (solid-state imaging element) side means a portion where the sheath 32b is peeled to expose the collective shield wire 32a and the cable.

The coaxial cable 33 is a coaxial cable in which the periphery of an inner conductor 33b is electromagnetically shielded by a shield wire 33a, has the inner conductor 33b, the insulator between the inner conductor 33b and the shield wire 33a, the shield wire 33a, and a sheath that covers the outer periphery of the shield wire 33a.

As illustrated in FIG. 2, FIG. 3, and the like, the coaxial cable 33 has the sheath peeled at the end part thereof on the image sensor 21 side to expose and the shield wire 33a and the inner conductor 33b.

A distal end part of the exposed inner conductor 33b is electrically connected to a connecting terminal 26a of the image sensor 21.

The single-axis cable 34 is a cable for supplying electrical power to the image sensor 21, and has an inner conductor 34a and a sheath that covers an outer periphery of the inner conductor 34a.

As illustrated in FIG. 2, FIG. 3, and the like, the single-axis cable 34 has the sheath peeled at an end part thereof on the image sensor 21 side to expose the inner conductor 34a.

A distal end part of the exposed inner conductor 34a is electrically connected to a connecting terminal 26b of the image sensor 21.

Although illustration is omitted, the cable bundle 32 may have a grounding cable for connecting the image sensor 21 to the ground, other coaxial cables, and the like, in addition to the above coaxial cable 33 and single-axis cable 34.

The cable bundle 32 is inserted into the insertion part and the universal cord of the endoscope, and the image sensor 21 is connected to the processor unit 4 via the plurality of cables of the cable bundle 32.

As a preferable aspect, the cable bundle 32 is inserted through the pipe-like member 30 in the vicinity of the end part thereof.

The pipe-like member 30 is a member that allows the cable bundle 32 to be inserted therethrough. Additionally, at least a portion of the pipe-like member 30 is disposed within the opening part 28a of the case 28.

In the example illustrated in FIG. 3, the pipe-like member 30 is a cylindrical member of which both ends are open, and the internal diameter thereof is a size such that the cable bundle 32 are insertable therethrough. That is, the pipe-like member 30 has an opening that is open at both ends and allows the cable bundle 32 to be inserted through. Additionally, in the example illustrated in FIG. 3, the pipe-like member 30 has a cylindrical shape and has a cutout part 30a at least in a portion of a peripheral surface thereof. In the example illustrated in FIG. 3, the pipe-like member 30 is disposed such that the direction of the cutout part 30a is aligned with the direction of the largest open surface of the case 28. Additionally, the pipe-like member 30 is disposed such that the cutout part 30a is located at a position where the collective shield wire 32a of the cable bundle 32 and the shield wire 33a of the coaxial cable 33 are exposed, in the longitudinal direction of the cable bundle 32.

Additionally, in the example illustrated in FIG. 3, at least a portion of the pipe-like member 30 is disposed in a portion (curved surface 28d) of the opening part 28a of the case 28 of which the cross-sectional shape is a substantially semicircular shape. The external diameter of the pipe-like member 30 substantially coincides with the diameter of the circle inscribed on the curved surface 28d, and a central axis of the external diameter of the pipe-like member 30 substantially coincides with the central axis 28C (refer to FIG. 7) of the circle inscribed on the curved surface 28d.

Although the pipe-like member 30 may be made of metals having conductivity or may be made of resins, it is preferable that the pipe-like member 30 has conductive metallic properties.

The pipe-like member 30 is joined to the case 28 in the portion of the through-hole 29 of the case 28. As methods of joining the pipe-like member 30 and the case 28 to each other, conductive joining methods using conductive joining members, such solder, metal braze, and metal paste, and methods, such as welding and pressure welding, are available. Additionally, the position where the case 28 and the pipe-like member 30 are joined to each other is also limited the bottom surface side of the case 28, and may be joined to each other at any positions as long as the case 28 and the pipe-like member 30 can be joined to each other.

In addition, in an example illustrated in FIG. 8, the cross-sectional shape of the portion (curved surface 28d) of the case 28 where the pipe-like member 30 of the opening part 28a is disposed is a substantially semicircular shape. However, the invention is not limited to this, and as illustrated in FIG. 10, the cross-sectional shape may be a step-like shape. In a case where the cross-sectional shape is the step-like shape, the diameter of an inscribed circle in contact with a top part of each step may be made to be substantially the same as the external diameter of the pipe-like member 30, and a central axis of the inscribed circle may be made to coincide with a central axis of the external diameter of the pipe-like member 30.

In addition, the cross-sectional shape of the outer shape of the pipe-like member 30 is not limited to the circular shape, and can be various shapes, such as a square shape, an oblong shape, and an elliptical shape. In this case, the cross-sectional shape of the outer shape of the pipe-like member 30 at the position where the pipe-like member 30 of the case 28 is installed may be a shape according to the cross-sectional shape of the curved surface 28d.

As a preferable aspect, as illustrated in FIG. 2, in the end part of the cable bundle 32 on the image sensor 21 side, the shield wire 33a of the coaxial cable 33 is covered with the conductive joining member 40b, and the collective shield wire 32a of the cable bundle 32 is covered with the conductive joining member 40a. The conductive joining members 40a and 40b are electrically joined to the pipe-like member 30 in the cutout part 30a of the pipe-like member 30. In addition, in the following description, in a case where the conductive joining members 40a and 40b do not need to be distinguished from each other, the conductive joining members 40 will be collectively described.

The conductive joining members 40 have at least one of solder, metal braze, and metal paste.

As the solder, various kinds of solder used for wiring boards or the like of related-art endoscopes are available.

As the metal braze, various kinds of metal braze used for wiring boards or the like of related-art endoscopes are available.

As the metal paste, various kinds of metal paste used for wiring boards or the like of related-art endoscopes are available.

By allowing the cable bundle to be inserted through the metallic pipe-like member and joining at least one of respective shield wires of the coaxial cable or the collective shield wire of the cable bundle to the pipe-like member by the conductive joining members in the cutout part of the pipe-like member, the shield wires and the collective shield wire can be protected without deterioration of the conductive joining members by chemicals.

Additionally, since the conductive joining members that fix the shield wires and the collective shield wire to each other are at least one of solder, metal braze, or the metal paste, the conductive joining members do not deteriorate easily due to chemicals. For that reason, even though disinfection and/or sterilization are repeated, a state where the shield wires and the collective shield wire are fixed to each other can be maintained. Therefore, it is possible to suppress a problem such that a cable may be pulled and load may be applied to a solder part of a cable end part to cause disconnection, in the case where the insertion part is curved.

Additionally, by joining the respective shield wires and the collective shield wire to each other by the conductive joining members, conduction between the shield wires can be ensured, and wiring is easy. By making electric potentials of the plurality of shield wires constant, the electromagnetic interference between the shield wires can be prevented, and noise can be reduced.

In addition, in the example illustrated in FIG. 2, a configuration in which the shield wire 33a of the coaxial cable 33 and the collective shield wire 32a of the cable bundle 32 are joined to the cutout part 30a of the pipe-like member 30 by the conductive joining members 40a and 40b, respectively, is adopted. However, the invention is not limited to this. A configuration in which the shield wire 33a of the coaxial cable 33 and the collective shield wire 32a of the cable bundle 32 are joined to the cutout part 30a of the pipe-like member 30 by one conductive joining member may be adopted.

Additionally, a configuration in which the cable bundle 32 has the four cables may be adopted. However, the invention is not limited to this. One to three cables may be provided or five or more cables may be provided. Additionally, the cable bundle 32 may have two or more coaxial cables.

Additionally, in the example illustrated in FIG. 2, a configuration in which the pipe-like member 30 has a cylindrical shape and has the cutout part 30a in a portion of the peripheral surface thereof is adopted. However, the invention is not limited to this as long as the shield wires are connectable by the conductive joining members. For example, a configuration having a cylindrical shape and a cutout part in the shape of a slit parallel to the longitudinal direction in a peripheral surface, that is, a substantially C-shaped member in which a cross-section perpendicular to the longitudinal direction, may be adopted.

Additionally, in the example illustrated in FIG. 2, the pipe-like member 30 has the cylindrical shape having the opening that allows the cable bundle 32 to be insertable therethrough. However, the invention is not limited to this as long as the cable bundle 32 can be held. For example, the pipe-like member may be a columnar member, or may have a shape having a slit that capable of holding the cable bundle 32 in a peripheral surface thereof. In other words, the pipe-like member may be a member in which a cross-sectional shape perpendicular to the longitudinal direction is substantially C-shaped.

Additionally, in the example illustrated in FIG. 2 and the like, a configuration in which the pipe-like member 30 and the case 28 are separately formed and the two members are joined to each other is adopted. However, the invention is not limited to this, and the pipe-like member 30 and the case 28 may be integrally formed.

Additionally, a configuration in which the shield wires of the coaxial cable and the collective shield wire of the cable bundle are covered with the conductive joining member 40 and resin mold is filled into the cutout part 30a of the pipe-like member 30 or the opening part 28a of the case may be adopted.

Additionally, it is preferable that materials of the pipe-like member 30, the case 28, and the holder are those having conductivity, such as stainless steel and brass.

Next, a method of manufacturing the endoscope of the invention will be described.

The method of manufacturing the endoscope of the invention is a method of manufacturing an imaging device to be used for an endoscope, and is a method of manufacturing an endoscope, having a joining step of joining a case and a holder to each other; an imaging element disposing step of disposing an imaging element so as to abut against at least one of inner wall surfaces of an opening part of the case and such that an image reception surface abuts against an end surface of a holder, after the joining step; and a lens assembly disposing step of disposing a lens assembly on the holder, in the joining step, the case and the holder are joined to each other in a state where a pin is inserted through the opening part of the case and a hole part of the holder and one opening surface of the opening part of the case abuts against an end surface of the holder, and a diameter of the pin, a diameter of the hole part of the holder, and a diameter of a circle inscribed on a cross-sectional shape in at least a portion of the opening part of the case coincide with each other.

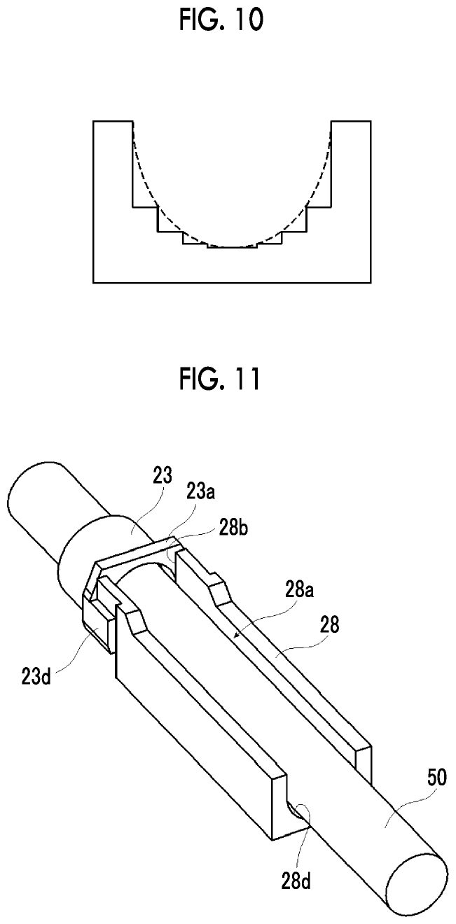

First, in the joining step, the case 28 and the holder 23 are joined to each other. In that case, as illustrated in FIG. 11, the case 28 and the holder 23 are joined to each other in a state where a columnar pin 50 is inserted through the opening part 28a of the case 28 and the hole part 23b of the holder 23, the curved surface 28d of the opening part 28a of the case 28 is made to abut against the pin 50, and the end surface 28b of the opening part 28a of the case 28 is made to abut against the end surface (flange part 23a) of the holder 23.

The external diameter of the pin 50 is substantially the same as the diameter of the hole part 23b of the holder 23, and is substantially the same as the diameter of the circle inscribed on the curved surface 28d of the case 28.

In addition, in a case where the diameter of the hole part 23b of the holder 23 is different from the diameter of the circle inscribed on the curved surface 28d of the case 28, the diameter of a portion of the pin 50 to be inserted through the hole part 23b of the holder 23 may be substantially the same as the diameter of the hole part 23b of the holder 23, and the diameter of a portion of the pin 50 to be inserted through the opening part 28a of the case 28 may be substantially the same as the diameter of the circle inscribed on the curved surface 28d of the case 28.

By performing positioning between the case 28 and the holder 23 using the pin 50, a central axis of the hole part 23b of the holder 23 and a central axis of the circle inscribed on the curved surface 28d of the case 28 can be made to coincide with each other with high accuracy. For that reason, the position in a surface direction (a surface direction of the surface where the holder 23 and the case 28 abut against each other) between the holder 23 and the case 28 can be positioned with high accuracy.

The joining between the holder 23 and the case 28 can be performed by methods using conductive joining members, such solder, metal braze, and metal paste, and well-known methods, such as welding and pressure welding.

After the holder 23 and the case 28 are joined to each other, the pin 50 is pulled out, and the holder 23 and the case 28 as illustrated in FIG. 9 are joined to each other.

Meanwhile, in a cable connecting step, the pipe-like member 30 is inserted through the distal end part of the cable bundle 32, and the inner conductor of each cable of the cable bundle 32 is connected to each connecting terminal 26 of the image sensor 21. The connection between the inner conductor and the connecting terminal 26 can be performed with solder, metal braze, metal paste, and the like.

Additionally, as a preferable aspect, the shield wires of each cable and the collective shield wire 32a of the cable bundle 32 are joined to the pipe-like member 30 in the cutout part 30a of the pipe-like member 30 by each conductive joining member 40.

Since the conductive joining member 40 is any of solder, metal braze and metal paste, the shield wires and the pipe-like member 30 may be joined to each other by the conductive joining member 40 by methods according to materials to be used.

In addition, after the shield wires and the collective shield wire 32a are joined to the pipe-like member 30 in the cutout part 30a of the pipe-like member 30 by the conductive joining member 40, the inner conductor of each cable may be connected to each connecting terminal 26 of the image sensor 21.

Next, in the imaging element disposing step, the image sensor 21 is disposed within the opening part 28a of the case 28. The image sensor 21 is disposed such that the image reception surface 21a is made to abut against the flange part 23a of the holder 23 and the lateral surfaces are made to abut against the inner wall surfaces of the case 28.

As mentioned above, by forming the surface for positioning the image sensor 21 such that the end surface 28b of the case 28 and the flange part 23a of the holder 23 are made to abut against each other, the rounded part is not formed between the end surface 28b of the case 28 and the flange part 23a of the holder 23. Therefore, the image reception surface 21a of the image sensor 21 can be thoroughly brought into contact with the flange part 23a of the holder 23, and the lateral surfaces of the image sensor 21 can be reliably brought into contact with the inner wall surface of the case 28. Therefore, the image reception surface of the image sensor can be positioned perpendicularly to the optical axis of the lens assembly, the center position of the image reception surface of the image sensor in a planar direction of the image reception surface can be positioned with high accuracy at the position of the optical axis of the lens assembly, and the distance between the image sensor and the lens assembly can be positioned with high accuracy. Therefore, the optical image to be formed by the lens assembly can be formed with high accuracy on the image reception surface of the imaging element, and a high-quality image with no loss of the image, defocusing, and the like can be obtained.

The case 28 and the image sensor 21 are preferably joined to each other with an adhesive.

The pipe-like member 30, which holds the cables connected to the connecting terminals 26 of the image sensor 21, is disposed on the curved surface 28d of the case 28. In that case, it is preferable to align the direction of the cutout part 30a of the pipe-like member 30 with the opening part 28a of the case 28.

As mentioned above, the joining between the case 28 and the pipe-like member 30 can be performed by the methods using the conductive joining members (solder, metal braze, and metal paste), and the methods, such as welding and pressure welding.

Next, in the lens assembly disposing step, the lens assembly 22 is disposed within the hole part 23b of the holder 23.

After the lens assembly 22 is moved in an axial direction within the hole part 23b and the distance thereof from the image sensor 21 is adjusted, the lens assembly 22 is fixed to the holder 23 by, for example, an adhesive or the like.

The imaging device 20 of the endoscope as illustrated in FIG. 2 is made by the above.

In addition, in the above example, a configuration in which the cable connecting step is performed before the imaging element disposing step is adopted. However, the invention is not limited to this. The cable connecting step may be performed after the imaging element disposing step.

Additionally, in the above example, a configuration in which the lens assembly disposing step is performed after the imaging element disposing step is adopted. However, the invention is not limited to this. The imaging element disposing step may be performed after the lens assembly disposing step.

Additionally, in a case where the cross-sectional shape of the hole part 23b of the holder 23 and/or the cross-sectional shape of curved surface 28d of the case 28 are other than the circular shape, the cross-sectional shape of the pin may be a shape according to these cross-sectional shapes.

Although the endoscope of the invention has been described above in detail, it is natural that the invention is not limited to the above aspects, and various improvements and modifications may be performed without departing from the scope of the invention.

### EXPLANATION OF REFERENCES

1: endoscope system
2: endoscope
3: light source unit
4: processor unit
20: imaging device
21: solid-state imaging element (image sensor)
21a: image reception surface
22: lens assembly
23: holder
23a: flange part
23b: hole part
23c: central axis
23d: claw part
26, 26a, 26b: connecting terminal
28: case
28a: opening part
28b: end surface
28c: central axis
28d: curved surface
29: through-hole
30: pipe-like member
30a: cutout part
32: cable bundle
32a: collective shield wire
32b: protective covering (sheath)
33: coaxial cable
33a: shield wire
33b: inner conductor
40a, 40b: conductive joining member
50: pin

What is claimed is:

1. An endoscope comprising:
an imaging element;
a lens assembly that forms an optical image on an image reception surface of the imaging element;
a cable that is connected to a connecting terminal provided on a surface opposite to the image reception surface of the imaging element;
a case that protects a joined part between the connecting terminal of the imaging element and the cable; and
a holder that holds the lens assembly and is joined to the case,
wherein the case has an opening part that penetrates in an arrangement direction of the imaging element and the cable, and is disposed such that an opening surface on the imaging element side is in contact with an end surface of the holder, and
wherein the imaging element is disposed such that a lateral surface abuts against at least one of inner wall surfaces of the opening part of the case and the image reception surface abuts against the end surface of the holder.

2. The endoscope according to claim 1,
wherein the lateral surface of the imaging element abuts against two adjacent inner wall surfaces of the opening part of the case.

3. The endoscope according to claim 1,
wherein the lateral surface of the imaging element abuts against two facing inner wall surfaces, and one inner wall surface adjacent to the two facing inner wall surfaces of the opening part of the case.

4. The endoscope according to claim 2,
wherein the lateral surface of the imaging element abuts against two facing inner wall surfaces, and one inner wall surface adjacent to the two facing inner wall surfaces of the opening part of the case.

5. The endoscope according to claim 1,
wherein the holder has at least one claw part to be locked to an outer wall surface of the case.

6. The endoscope according to claim 4,
wherein the holder has at least one claw part to be locked to an outer wall surface of the case.

7. The endoscope according to claim 1,
wherein the holder has a hole part that penetrates in the arrangement direction of the imaging element and the cable and holds the lens assembly.

8. The endoscope according to claim 6,
wherein the holder has a hole part that penetrates in the arrangement direction of the imaging element and the cable and holds the lens assembly.

9. The endoscope according to claim 7, further comprising:
a cylindrical pipe-like member into which the cable is to be inserted,
wherein the pipe-like member is disposed within the opening part of the case, and
wherein a central axis of a circle inscribed on a cross-sectional shape of the opening part of the case at a disposition position of the pipe-like member, a central axis of an external diameter of the pipe-like member, and a central axis of the hole part of the holder coincide with each other.

10. The endoscope according to claim 8, further comprising:
a cylindrical pipe-like member into which the cable is to be inserted,
wherein the pipe-like member is disposed within the opening part of the case, and
wherein a central axis of a circle inscribed on a cross-sectional shape of the opening part of the case at a disposition position of the pipe-like member, a central axis of an external diameter of the pipe-like member, and a central axis of the hole part of the holder coincide with each other.

11. The endoscope according to claim 9,
wherein a diameter of the circle inscribed on the cross-sectional shape of the opening part of the case at the disposition position of the pipe-like member and the external diameter of the pipe-like member coincide with each other.

12. The endoscope according to claim 10,
wherein a diameter of the circle inscribed on the cross-sectional shape of the opening part of the case at the disposition position of the pipe-like member and the external diameter of the pipe-like member coincide with each other.

13. The endoscope according to claim 1,
wherein the cross-sectional shape of the opening part of the case at the disposition position of the imaging element is a rectangular shape.

14. The endoscope according to claim 12,
wherein the cross-sectional shape of the opening part of the case at the disposition position of the imaging element is a rectangular shape.

15. A method of manufacturing the endoscope according to claim 1, comprising:
a joining step of joining the case and the holder to each other;
an imaging element disposing step of disposing the imaging element such that the imaging element abuts against at least one of inner wall surfaces of the opening part of the case and the image reception surface abuts against an end surface of the holder, after the joining step; and
a lens assembly disposing step of disposing the lens assembly on the holder,
wherein in the joining step, the case and the holder are joined to each other in a state where a pin is inserted through the opening part of the case and a hole part of the holder and one opening surface of the opening part of the case abuts against an end surface of the holder, and
wherein a diameter of the pin, a diameter of the hole part of the holder, and a diameter of a circle inscribed on a cross-sectional shape in at least a portion of the opening part of the case coincide with each other.

16. A method of manufacturing the endoscope according to claim 14, comprising:
a joining step of joining the case and the holder to each other;
an imaging element disposing step of disposing the imaging element such that the imaging element abuts against at least one of inner wall surfaces of the opening part of the case and the image reception surface abuts against an end surface of the holder, after the joining step; and
a lens assembly disposing step of disposing the lens assembly on the holder,
wherein in the joining step, the case and the holder are joined to each other in a state where a pin is inserted through the opening part of the case and a hole part of the holder and one opening surface of the opening part of the case abuts against an end surface of the holder, and
wherein a diameter of the pin, a diameter of the hole part of the holder, and a diameter of a circle inscribed on a cross-sectional shape in at least a portion of the opening part of the case coincide with each other.

17. The method of manufacturing the endoscope according to claim 15, further comprising:
a cable connecting step of connecting the cable to the connecting terminal of the imaging element before the imaging element disposing step.

18. The method of manufacturing the endoscope according to claim 16, further comprising:
a cable connecting step of connecting the cable to the connecting terminal of the imaging element before the imaging element disposing step.
